(19) United States
(12) Patent Application Publication
(10) Pub. No.: US 2020/0113517 A1
(43) Pub. Date: Apr. 16, 2020

(54) METHOD FOR AUTOMATICALLY IDENTIFYING USERS OF BODY-FAT METER

(71) Applicant: CAL-COMP BIG DATA, INC., New Taipei City (TW)

(72) Inventor: Hung-TAI HSU, NEW TAIPEI CITY (TW)

(21) Appl. No.: 16/371,058

(22) Filed: Mar. 31, 2019

(30) Foreign Application Priority Data
Oct. 11, 2018 (CN) 201811183689.0

(51) Int. Cl. A61B 5/00; G16H 40/63; G01G 19/50; A61B 5/117

(52) U.S. Cl. CPC A61B 5/4872; A61B 5/117; G01G 19/50; G16H 40/63

(57) ABSTRACT

A method for automatically identifying users of a body-fat meter includes following steps: measuring body information of a user through a measuring unit of the body-fat meter; obtaining multiple user accounts registered in a memory; respectively reading a personal data and multiple historical body values relative to each user account; calculating a predicted value for each user account based on the measured body information and the personal data of each user account; determining if any of the user accounts has the multiple historical body values that are matching with its corresponding predicted value; and, storing the corresponding predicted value for the matched user account for updating the multiple historical body values of the user account.

### BACKGROUND OF THE INVENTION

### 1. Technical Field

The technical field relates to a body-fat meter, and specifically relates to a method for identifying users of the body-fat meter.

### 2. Description of Related Art

Body-fat meters can measure a bunch of user information such as weight and body-fat, and in cooperate with height information of a user, the body-fat meters can also calculate user's body mass index (BMI) information. In conclusion, the body-fat meters are popular to the normal families.

For calculating the aforementioned BMI information, a user usually has to manually input the height information to a body-fat meter by himself/herself, or uses his/her personal account to login to the body-fat meter in advance, so the body-fat meter can retrieve necessary data for calculating BMI information. However, the user has to do manual procedure(s) before using the body-fat meter (i.e., inputs data or selects account), which is inconvenient.

For the purpose of inputting and displaying, parts of the body-fat meters and weight scales in the market have to arrange an input unit and a monitor on the shell, which affects their production cost, size and appearing design. Furthermore, to perform data input or login to the body-fat meter is also bothering the users, therefore, a method for the body-fat meters which can automatically identify user identity after finishing the measurement will be really helping and may increase the convenience of using the body-fat meters.

### SUMMARY OF THE INVENTION

The invention is directed to a method for automatically identifying users of body-fat meter, which can automatically identify user identity right after the measurement of the user is finished.

In one of the exemplary embodiments of the present invention, the above method is adopted by a body-fat meter and includes following steps: measuring and obtaining body information of a user through a measuring unit of the body-fat meter; obtaining one or more registered user accounts from a memory; reading personal data and multiple historical body values from each of the one or more user accounts; calculating multiple predicted values respectively in accordance with the body information and the personal data of each of the one or more user accounts, wherein each of the predicted values is respectively corresponding to each of the one or more user accounts; determining whether each of the one or more user accounts having the multiple historical body values which are matching with its corresponding one of the predicted values; and, if any one of the one or more user accounts is determined having the multiple historical body values matching with its corresponding predicted value, storing the corresponding predicted value to this user account for updating the multiple historical body values of this specific user account.

In comparison with related arts, the technical effect of the present invention is that the user does not have to manually select any account and login to the body-fat meter before conducting a measurement on the body-fat meter, and the body-fat meter may automatically identify user identity right after the measurement of the user is done, which optimizes the measuring procedure and increases the convenience in using the body-fat meter.

### BRIEF DESCRIPTION OF THE DRAWINGS

FIG. 1 is schematic diagram of a first embodiment showing a body-fat meter of the present invention.

FIG. 2 is a block diagram of a first embodiment showing the body-fat meter of the present invention.

FIG. 3 is a first identifying flowchart of a first embodiment for the body-fat meter of the present invention.

FIG. 4 is a second identifying flowchart of a first embodiment for the body-fat meter of the present invention.

FIG. 5 is a schematic diagram of a first embodiment showing an alarm of the present invention.

FIG. 6 is a schematic diagram of a second embodiment showing an alarm of the present invention.

### DETAILED DESCRIPTION OF THE INVENTION

In cooperation with the attached drawings, the technical contents and detailed description of the present invention are described thereinafter according to multiple embodiments, being not used to limit its executing scope. Any equivalent variation and modification made according to appended claims is all covered by the claims claimed by the present invention.

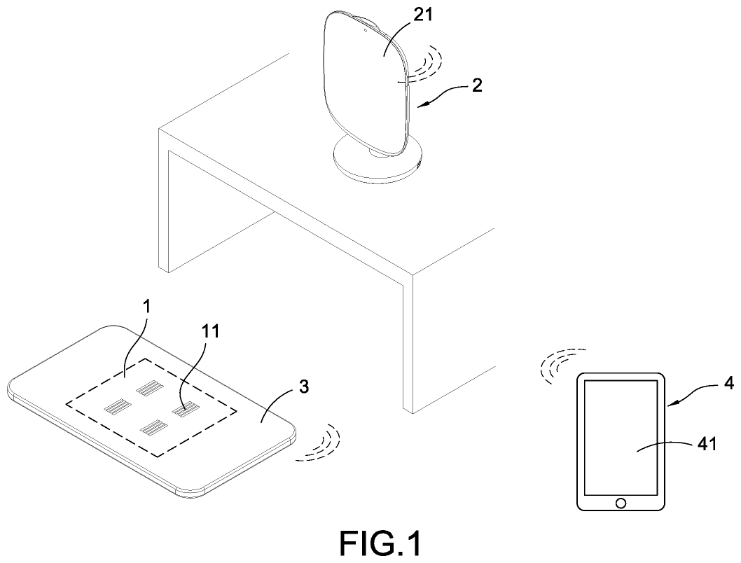

FIG. 1 is schematic diagram of a first embodiment showing a body-fat meter of the present invention. The present invention discloses a method for automatically identifying users of body-fat meter (referred to as the identifying method hereinafter), the identifying method is adopted by a smart body-fat meter 1 (referred to as the body-fat meter 1 hereinafter). In one embodiment, all data relative to the body-fat meter 1 are stored in the body-fat meter 1, and each step of the identifying method described below are executed by the body-fat meter 1. In other embodiment, all data relative to the body-fat meter 1 may be stored in an electronic device (such as a smart mirror device 2 or a mobile device 4) or a cloud server through wired or wireless matters, and each step of the identifying method described below may be executed by the electronic device or/and the cloud server in a non-limiting way.

For the sake of understanding, the descriptions below will take the body-fat meter 1 storing relative data and executing necessary steps for a main example, but not intended to narrow down the claimed scope of the present invention.

As disclosed in the embodiment of FIG. 1, the body-fat meter 1 is basically for house using and can be hidden in the decoration of the house, such as a carpet 3, floor, etc. . . . . In such way, the user doesn't have to store the body-fat meter 1 after measuring, and it won't affect the indoor space of the house, which may increase the flexibility of using the body-fat meter 1. When the user intends to use the body-fat meter 1 to measure body information (such as weight, body-fat, etc.), he or she only have to stand on a measuring unit 11 of the body-fat meter 1 that is revealed from the decoration and the measuring procedure will be instantly activated, which is very convenient.

For reducing the volume of the body-fat meter 1 so it can be assimilated to the decoration with ease, the body-fat meter 1 of the present embodiment can be arranged without any monitor. In particular, the body-fat meter 1 may be communicated or connected with an external electronic device via wired or wireless matters, and it may display data relative to the body-fat meter 1 through the electronic device which is connected thereto. For example, the body-fat meter 1 may display data through a displaying unit 21 of the smart mirror device 2 or a displaying screen 41 of the mobile device 4.

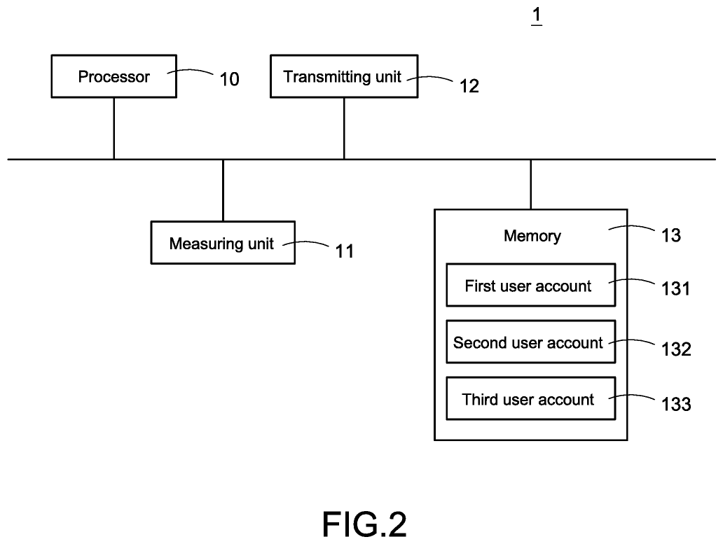

FIG. 2 is a block diagram of a first embodiment showing the body-fat meter of the present invention. As shown in FIG. 2, the body-fat meter 1 mainly includes a processor 10, and also includes a measuring unit 11, a transmitting unit 12 and a memory 13 which are electrically connected with the processor 10. The measuring unit 11 is used to physically contact with the user for measuring each body information of the user, such as weight, body-fat, etc. . . . . The processor 10 obtains the body information measured by the measuring unit 11, and performs further calculation to the body information according to personal data of the user, so as to generate different types of body value for the user, such as body mass index (BMI), basal metabolic rate (BMR), etc. . . . .

In another embodiment, the body-fat meter 1 may measure the aforementioned body information of the user by the measuring unit 11, and transmits the measured body information to the external electronic devices (such as the smart mirror device 2, the mobile device 4 or the cloud server) through the transmitting unit 12, so as to calculate the aforementioned body value(s) of the user by the electronic device.

In the embodiment, the body-fat meter 1 may accept the registration from one or more users in advance, create one or more user accounts respectively corresponding to the one or more users, and store the one or more user accounts in the memory 13. In particular, each of the one or more user accounts respectively may record personal data and multiple historical body values of the corresponding user. In the embodiment of

FIG. 2, the memory 13 stores multiple user accounts including a first user account 131 corresponding to a first user, a second user account 132 corresponding to a second user and a third user account 133 corresponding to a third user, but the number of the user accounts is not limited to what is disclosed in FIG. 2.

Before the identifying method of the present invention is activated by the body-fat meter 1, the user may manually select one of the multiple user accounts stored in the body-fat meter 1 for logging to the body-fat meter 1 before conducting a measuring procedure, and then the user can measure his/her body information through the measuring unit 11 of the body-fat meter 1. Next, the processor 10 of the body-fat meter 1 may calculate each of the aforementioned body values for the user, and store the calculated body values to the selected user account for updating the multiple historical body values of this user account.

Following the above description, when usage count or usage time of the user account reaches a default threshold (for example, the usage count reaches ten times, or the usage time reaches one month), the body-fat meter 1 may activate the identifying method of the present invention for assisting the user (i.e., the owner of this user account) in using the body-fat meter 1 cooperated with an intellectual calculation.

In other embodiments, the aforementioned user accounts 131-133 may also be stored in the smart mirror device 2, the mobile device 4 or the cloud server, not only limited to what is disclosed in FIG. 2. For the sake of understanding, the memory 13 of the body-fat meter 1 will be taken as an example for storing these user accounts 131-133.

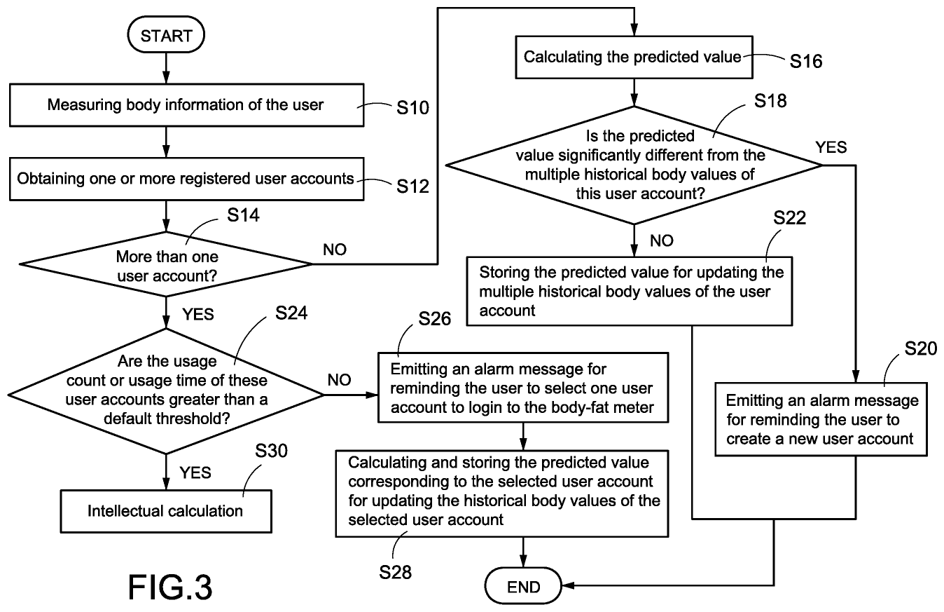

FIG. 3 is a first identifying flowchart of a first embodiment for the body-fat meter of the present invention. First, a user may stand on the body-fat meter 1 when he or she intends to conduct a measuring procedure of the body-fat meter 1, and the body-fat meter 1 measures body information of the user through the measuring unit 11 (step S10). In the embodiment, the body information may be, for example, weight, body-fat, or other information that can be measured by the body-fat meter 1.

Next, the processor 10 of the body-fat meter 1 obtains one or more registered user accounts from the memory 13 (step S12), and respectively reads the personal data and the multiple historical body values of each of the user accounts. In the embodiment, the personal data may be the basic data of the user, such as gender, height, age, etc. that had been pre-stored in the body-fat meter 1, and the historical body values may be, for example, weight, body-fat, muscle rate, BMI, BMR, etc. of the user that had been measured and calculated in the past, but not limited thereto.

One of the main technical features of the present invention is that the processor 10 may conduct a calculation according to the measured body information of the user and the personal data of each of the user accounts, so as to respectively generate a predicted value for each of the user accounts. For example, if ten user accounts (owned by ten different users) are stored in the body-fat meter 1, the processor 10 will calculate ten predicted values that are respectively corresponding to each of the ten user accounts. Also, the processor 10 determines which user account is corresponding to the user who's currently measuring through these predicted values. In one embodiment, the data type of the predicted values is the same as that of the historical body values (such as BMI or BMR).

After the step S12, the processor 10 determines whether the memory 13 stores more than one user account (step S14). If the memory 13 stores only one user account, the processor 10 will directly read the personal data of this user account, and calculates one predicted value corresponding to this user account based on the measured body information and the personal data of this user account (step S16), and then determines whether the predicted value is significantly different from the multiple historical body values of this user account (step S18). In one embodiment, if the predicted value is determined having significant difference with the historical body values of this user account, then the differences between the predicted value and the historical body values will be greater than a tolerable error threshold. When the predicted value is significant different from the historical body values of this user account, the processor 10 may determine that the predicted value is not belonging to the user who owns this user account.

For example, the processor 10 may divide the measured weight of the user with the height recorded in the personal data of the user account, so as to obtain the predicted value of the user (which is BMI). If the predicted value is 20, but the multiple historical body values (is BMI as well) of the user account are all greater than 24, the processor 10 may determine that the predicted value is significantly different from the multiple historical body values of the user account.

In one embodiment, the processor 10 determines that the predicted value is significantly different from the historical body values of the user account when the differences between the predicted value and the historical body values are greater than a threshold (such as 3). In another embodiment, the processor 10 may determine that the predicted value is significantly different from the historical body values of the user account if the measured body information of the user (such as weight) and the historical body values (such as weight) are belonging to different intervals (for example, an underweight interval, a moderate weight interval, an overweight interval, etc.), but not limited thereto.

If the processor 10 determines positive in the step S18 (which indicates that the predicted value is significantly different from the historical body values of the user account), it means the user who's currently measuring by the body-fat meter 1 may not be the owner of this user account, therefore, the processor 10 may emit an alarm message for reminding the current user to create a new user account (step S20). In this embodiment, the processor 10 may send a control command to the connected smart mirror device 2 or the mobile device 4, so as to emit the alarm message and accept an operation from the user for creating a new user account through the smart mirror device 2 or the mobile device 4, in a non-limiting way.

If the processor 10 determines negative in the step S18 (which indicates that the predicted value is not significantly different from the historical body values of the user account), it means the user who's currently measuring by the body-fat meter 1 may be the owner of the user account, so the processor 10 directly stores the predicted value to this user account for updating the multiple historical body values of the user account (step S22). By way of the aforementioned measuring procedure, no matter a user has registered an account to the body-fat meter 1 before measuring or not, he or she may directly use the body-fat meter 1 to conduct a measurement anyway, which is very convenient.

If the processor 10, in the step 14, determines that the memory 13 stores multiple different user accounts, it should then determine whether the usage count or usage time of these user accounts are greater than a default threshold (step S24). In other words, the processor 10 has to determine if the one or more historical body values of these user accounts that has been stored in the memory 13 are qualified for the body-fat meter 1 to accomplish the intellectual calculation of the present invention or not.

If the usage count or the usage time of these user accounts are not greater than the threshold, it means that the processor 10 may not able to correctly determine which one of the multiple user accounts in the memory 13 is corresponding to the current user. In such scenario, the processor 10 may emit an alarm message for reminding the current user to manually select one of the multiple user accounts to login to the body-fat meter 1 (step S26). After the user selects one of the multiple user accounts, the processor 10 may read the personal data of the selected user account, and calculates a predicted value that is corresponding to the selected user account based on the measured body information and the personal data, and stores this predicted value to the selected user account for updating the one or more historical body values of the selected user account (step S28).

On the contrary, if the usage count or usage time of these user accounts are greater than the threshold, it means that the processor 10 should be able to correctly determine which one of the multiple user accounts is corresponding to the current user. In such scenario, the processor 10 may conduct the intellectual calculation in company with the measured body information (step S30), so as to automatically identify the identity of the user who's currently using the body-fat meter 1.

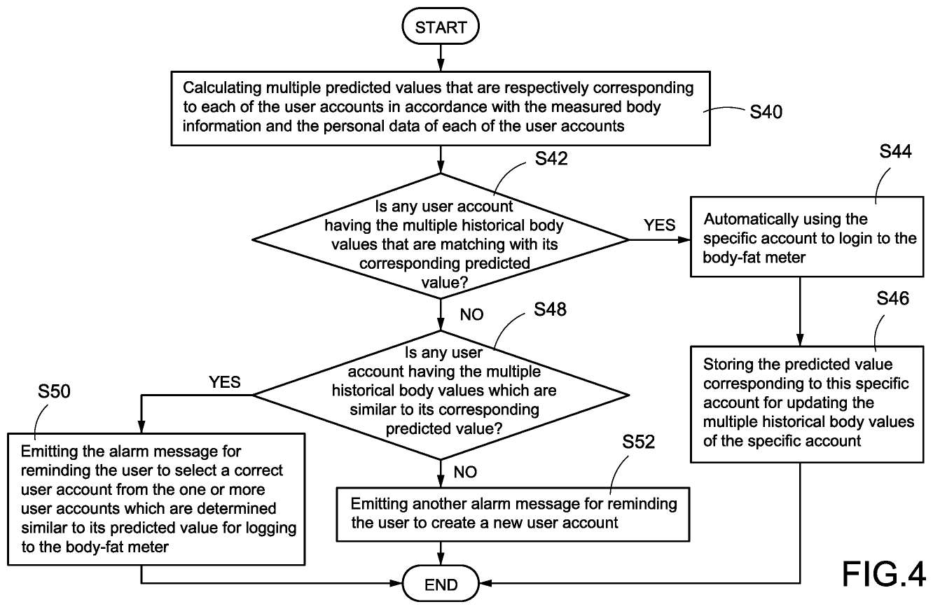

FIG. 4 is a second identifying flowchart of a first embodiment for the body-fat meter of the present invention. FIG. 4 is used for detailed describing the intellectual calculation adopted by the identifying method of the present invention.

As shown in FIG. 4, when the body-fat meter 1 activates the intellectual calculation, the processor 10 may calculate multiple predicted values that are respectively corresponding to each of the user accounts in accordance with the measured body information and the personal data of each of the user accounts (step S40), and the processor 10 determines that if any of the user accounts has the multiple historical body values that are matching with its corresponding predicted value (step S42). In one embodiment, the processor 10 may calculate a standard deviation and an average value of the multiple historical body values of one user account, and then determines whether the corresponding predicted value is matching with the multiple historical body values based on the standard deviation (or its multiple) and the average value.

For an example, assuming that a user account has stored four historical body values (such as BMI) which are 5,6,8,9, the standard deviation of these four values is 1.58, and the average value of these four values is 7. The processor 10 may then determine if a predicted value corresponding to this user account is matching with the existing four historical body values according to the standard deviation and the average value.

For another example, the first user account 131 may include a first personal data and ten first historical body values. The processor 10 may calculate a first predicted value (such as BMI) corresponding to the first user account 131 based on the measured body information (such as weight) and the first personal data (such as height), and determines if the first predicted value is matching with the ten first historical body values (for example, determines if the first predicted value is identical to a sum or a difference of the standard deviation (or its multiple) and the average value of the ten first historical body values, or if a variation of the first predicted value and the sum or the difference is smaller than a first threshold). For another example, the second user account 132 may include a second personal data and fifteen second historical body values. The processor 10 may calculate a second predicted value corresponding to the second user account 132 based on the measure body information and the second personal data, and determines if the second predicted value is matching with the fifteen second historical body values, and so on.

The aforementioned data calculating and comparing can be further represented by the following table:

| | | Calculating | |
|---|---|---|---|
| Account Name | Data in the Account | Result | Comparing Result |
| First User | First Personal Data and | First Predicted | Matched or Unmatched |
| Account | multiple First Historical | Value | |
| | Body Values | | |
| Second User | Second Personal Data and | Second | Matched or Unmatched |
| Account | multiple Second Historical | Predicted Value | |
| | Body Values | | |
| Third User | Third Personal Data and | Third Predicted | Matched or Unmatched |
| Account | multiple Third Historical | Value | |
| | Body Values | | |

If the processor 10 determines, in the step S42, that at least one of the multiple user accounts has the multiple historical body values that are matching with its corresponding predicted value, it will then store the predicted value to the corresponding user account for updating the multiple historical body values of this user account. In particular, when the processor 10 determines that one of the multiple user accounts (such as a specific account) has the multiple historical body values that are matching with its corresponding predicted value, it will automatically use this specific account to login to the body-fat meter 1 (step S44), and the processor 10 may then store this predicted value to this specific account for updating the multiple historical body values of this specific account (step S46).

In the step S44 and the step S46, the processor 10 determines that the user who's currently measuring by the body-fat meter 1 is the owner of the specific account, so the processor 10 automatically stores the measured body information (such as weight, body-fat, etc.) and the calculated predicted value (such as BMI) to the specific account for updating the specific account. As a result, the user doesn't have to manually select his/her own account for logging to the body-fat meter 1, instead, the user may instantly use the body-fat meter 1 and conduct the measuring procedure without any manual operation, which is very convenient.

If the processor 10, in the step S42, determines that none of the multiple user accounts in the memory 13 has the multiple historical body values which may be matching with its corresponding predicted value, it then determines if any of the multiple user accounts has the multiple historical body values which are similar to its corresponding predicted value (step S48).

In this embodiment, if the difference between the multiple historical body values of a user account and its corresponding predicted value is smaller than a first threshold, the processor 10 will determine that the multiple historical body values of this user account are matching with its corresponding predicted value. If the difference between the multiple historical body values of a user account and its corresponding predicted value is greater than the first threshold but smaller than a second threshold, the processor 10 will determine that the multiple historical body values of this user account and its corresponding predicted value are not matching, but similar. In the embodiment, the second threshold is greater than the first threshold.

If the processor 10 determines, in the step S48, that one or more of the multiple user accounts do have the multiple historical body values that are similar to its corresponding predicted value, it may emit an alarm message for confirming the identity of the user. In particular, the processor 10 may emit the alarm message through the electronic device for reminding the user to select a correct user account from the one or more user accounts which are determined similar to its predicted value, so as to login to the body-fat meter 1 (step S50).

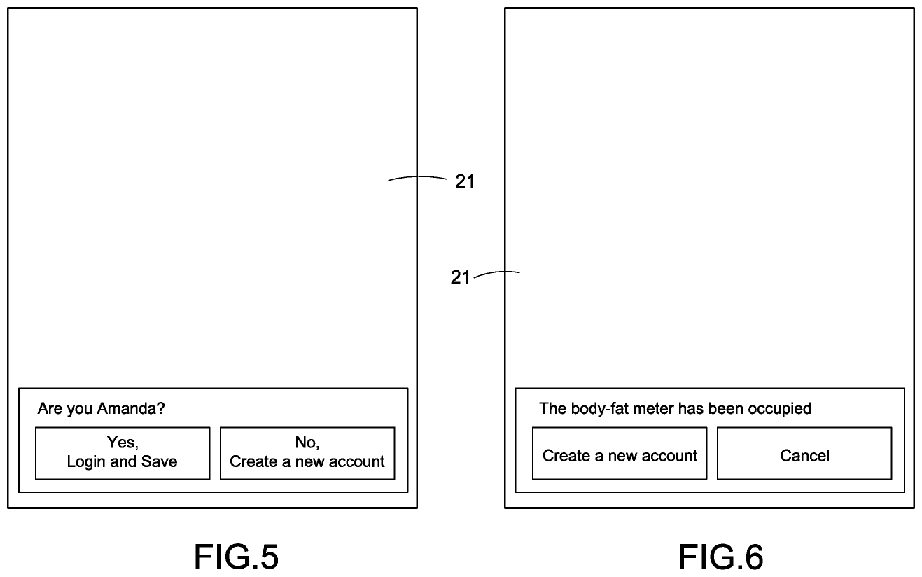

Please refer to FIG. 5, which is a schematic diagram of a first embodiment showing an alarm of the present invention. If the processor 10 determines that the measured body information is similar to one of the registered user accounts (but not totally matched), the processor 10 may display this similar user account for the user to confirm through the electronic device (for example, displays the similar user account via the displaying unit 21 of the smart mirror device 2). If the user account is confirmed by the user, he or she may use the confirmed user account to login to the body-fat meter 1 for storing data. If the user account is incorrect, the user may choose to create a new user account.

In the embodiment shown in FIG. 5, the body-fat meter 1 displays one user account (for example, belongs to Amanda) which is determined similar to the measured body information. In another embodiment, the body-fat meter 1 may simultaneously display multiple user accounts which are all determined similar to the measured body information for the user to confirm, not limited thereto.

Please refer back to FIG. 4. If the processor 10 determines, in the step S48, that none of the user accounts in the memory 13 has the historical body values that are similar to its corresponding predicted value, it may emit another alarm message for reminding the user to create a new user account (step S52). In particular, the processor 10 determines that all the registered user accounts of the body-fat meter 1 are not belonging to the user who's currently measuring by the body-fat meter 1, so the processor 10 requests the user to create a new account for storing the measured data of the user.

FIG. 6 is a schematic diagram of a second embodiment showing an alarm of the present invention. If the processor 10 determines that all of the registered user accounts of the memory 13 are neither matched nor similar to the measured body information of the user, it may display a menu for creating a new user account on the electronic device (such as the displaying unit 21 of the smart device 2), so the user may create a new user account with ease for logging to the body-fat meter 1 and storing the measured data.

By using the identifying method of the present invention, a user may use the body-fat meter to conduct a measuring procedure instantly no matter the user has registered an account to the body-fat meter yet or not, which is very convenient.

As the skilled person will appreciate, various changes and modifications can be made to the described embodiment. It is intended to include all such variations, modifications and equivalents which fall within the scope of the present invention, as defined in the accompanying claims.

What is claimed is:

1. A method for automatically identifying users of body-fat meter, adopted by a body-fat meter and comprising following steps:
a) measuring and obtaining body information of a user through a measuring unit of the body-fat meter;
b) obtaining one or more registered user accounts from a memory;
c) reading personal data and multiple historical body values from each of the one or more user accounts;
d) calculating multiple predicted values respectively in accordance with the body information and the personal data of each of the one or more user accounts, wherein each of the predicted values is respectively corresponding to each of the one or more user accounts;
e) determining whether each of the one or more user accounts having the multiple historical body values which are matching with its corresponding one of the predicted values; and
f) if any one of the one or more user accounts is determined having the multiple historical body values matching with its corresponding predicted value, storing the corresponding predicted value to this user account for updating the multiple historical body values of this specific user account.

2. The method for automatically identifying users of body-fat meter in claim 1, wherein the personal data at least comprises gender, height and age.

3. The method for automatically identifying users of body-fat meter in claim 1, wherein the predicted values and the historical body values are body mass index (BMI) or basal metabolic rate (BMR).

4. The method for automatically identifying users of body-fat meter in claim 1, wherein the step e) is to calculate a standard deviation and an average value of the multiple historical body values, use the standard deviation or the multiple of the standard deviation and the average value as references for determining whether the multiple historical body values are matching with the corresponding predicted value.

5. The method for automatically identifying users of body-fat meter in claim 1, wherein the step f) comprises following steps:
f1) automatically logging to the body-fat meter by using a specific user account after determining that the specific user account of the one or more user accounts having the multiple historical body values that are matching with its corresponding predicted value; and
f2) storing the corresponding predicted value to the specific user account for updating the multiple historical body values of the specific user account.

6. The method for automatically identifying users of body-fat meter in claim 1, further comprising following steps:
g) determining whether any of the one or more user accounts having the multiple historical body values that are similar to its corresponding predicted value after determining that none of the one or more user accounts having the multiple historical body values that are matching with its corresponding predicted value;
h) if one or more of the one or more user accounts are determined having the multiple historical body values that are similar to its corresponding predicted value, emitting a first alarm message for reminding the user to select a correct user account from the one or more user accounts that are determined similar to its predicted value to login to the body-fat meter; and
i) emitting a second alarm message for reminding the user to create a new user account after determining that none of the one or more user accounts having the multiple historical body values that are similar to its corresponding predicted value.

7. The method for automatically identifying users of body-fat meter in claim 1, further comprising following steps after the step b):
b1) determining if the memory stores multiple different user accounts;
b2) executing the step c) to the step f) if the memory stores multiple user accounts;
b3) if the memory stores only one user account, reading the personal data and the multiple historical body values of the user account;
b4) calculating the predicted value corresponding to the user account based on the personal data of the user account and the measured body information following the step b3);
b5) determining whether the predicted value is significantly different from the multiple historical body values of the user account following the step b4);
b6) emitting a third alarm message for reminding the user to create a new user account when determining that the predicted value is significantly different from the multiple historical body values of the user account; and
b7) storing the predicted value to the user account for updating the multiple historical body values of the user account when determining that the predicted value is not significantly different from the multiple historical body values of the user account.

8. The method for automatically identifying users of body-fat meter in claim 7, wherein the step b2) comprises following steps:
b21) if the memory stores multiple user accounts, determining whether a usage count or a usage time of each of the multiple user accounts is greater than a threshold;
b22) executing the step c) to the step f) if the usage count or the usage time of each of the multiple user accounts is greater than the threshold;
b23) emitting a fourth alarm message for reminding the user to select one of the multiple user accounts for logging to the body-fat meter if the usage count or the usage time of each of the multiple user accounts is not greater than the threshold; and
b24) storing the predicted value corresponding to the selected user account for updating the multiple historical body value of the selected user account.

9. The method for automatically identifying users of body-fat meter in claim 1, wherein the body-fat meter is communicated with a smart mirror device, the memory is arranged in the smart mirror device, and the smart mirror device has a displaying unit for displaying the one or more user accounts, the personal data, the multiple historical body values and the predicted values.

10. The method for automatically identifying users of body-fat meter in claim 1, wherein the body-fat meter is communicated with a mobile device, the memory is arranged in the mobile device, and the mobile device has a displaying screen for displaying the one or more user accounts, the personal data, the multiple historical body values and the predicted values.
